(19) United States
(12) Patent Application Publication
(10) Pub. No.: US 2012/0123252 A1
(43) Pub. Date: May 17, 2012

(54) IMAGING APPARATUS FOR LARGE AREA IMAGING OF A BODY PORTION

(75) Inventor: Wolfgang Brunner, Isny (DE)

(73) Assignee: ZEBRIS MEDICAL GMBH, Isny im Allgau (DE)

(21) Appl. No.: 13/296,827

(22) Filed: Nov. 15, 2011

(30) Foreign Application Priority Data
Nov. 16, 2010 (DE) 10 2010 051 519.1

(51) Int. Cl. A61B 6/00

(52) U.S. Cl. 600/425

(57) ABSTRACT

An imaging apparatus for large area imaging of a body portion of a vertebrate, having an optical scanner for generating image data, an image data processing unit for generating a primary skeleton image of the bone and/or joint structure from the image data of the body surface and stored standard skeleton images. A sensing head scans a depth scan image of the body portion stepwise or a sensing device senses bony references in the body portion. Markers mark the position and orientation of the sensing head or device for spatial orientation, and a navigation base station records spatial positions of the markers. A scanning data-processing unit produces a corrected skeleton image and/or anatomical rotational points of the body portion based on the primary skeleton image and on the depth scan image provided with position data and/or the position data of the bony references scanned with the sensing device.

### CROSS-REFERENCE TO RELATED APPLICATIONS

This application claims the benefit of German Patent Application No. DE 10 2010 051 519.1, filed Nov. 16, 2010, which is incorporated herein by reference as if fully set forth.

### BACKGROUND

The present invention relates to an imaging apparatus for the area imaging of a body portion of a vertebrate, in particular the back of a person, also known as a 3D-back scanner.

Such apparatus have been known for a long time and are offered, among other things, by the Applicant. Information concerning apparatus of this type is found, for example, under www.diers.de. Usually, structured light patterns are projected onto the back. From the 3D surface, specific features, e.g. the lumbar dimple can be determined and various parameters derived. It is endeavored, among other things, to draw conclusions on bony structures on the basis of the skin surface (e.g. location and form of the spinal column or of the pelvis). If features are not recognizable on the skin surface, then marking points are adhered and used for further determination. A disadvantage of this method is that the skin surface and the underlying soft tissues are very movable and depending to the constitution of the patient are recorded very indistinctly and therefore, significant uncertainty exists when reconstructing bony structures.

Well known and in clinical use, on the other hand, are systems for determining the position of body sections or parts of vertebrates, also designated as navigation systems. Reference is made from the extensive patent literature on these systems in view of the present solution in particular to DE 196 49 399 A1 and to DE 103 10 331 B3 of the Applicant, both of which disclose special apparatus for measuring the mobility of the back, respectively, the functional segments of the spinal column of a person. In DE 196 49 399 A1, brackets with marking means attached with belts to the back of a person are used for this purpose, permitting an accurate and also dynamic positioning of the respective body section based on the running time measurements in cooperation with a stationary measuring station. DE 103 10 331 B3 teaches the use of an ultrasound sensing head provided with similar marking means for examining the back of a patient whose exact spatial position is determined according to the same principle. This system enables in particular the generation of precise images of the surface contours from spinous and transversal processes of the spinal column and ultimately a determination of their mobility.

### SUMMARY

The object of the invention is to provide an improved apparatus of the type of the mentioned 3D back scanner which enables in particular the generation of more exact images of (especially bony) structures below the body surface and therewith an improved diagnostic information.

This objective is met by an imaging apparatus with the features of the invention. Useful embodiments of the inventive concept are described below and in the claims.

The proposed apparatus comprises first of all—in a manner known per se—a large area imaging, in particular an optical, scanning means for generating image data of the body surface and an image data processing unit for generating a primary skeleton image of the bone structure of the body section from the image data of the body surface and stored standard skeleton images. In addition to optical scanning devices such as the 3D back scanners known in this field or also laser scanners or high resolution cameras, for the generation of large area imaging also imaging methods using ultrasound or infrared cameras or also mechanical sensing devices are considered whose data sets can be composed to an overall image of the body surface of interest. Standard skeleton images or respectively, corresponding anatomical data sets are available in medical databases and can be stored internally for use in the proposed apparatus, or also patient-specific data sets (which were generated through other image generating methods) can be stored and used.

In addition, the apparatus includes a sensing head for the step-wise scanning of a depth scan image of the body portion and/or a sensing device for sensing bony references, respectively, joint points in the body portion and marking means for marking the position and orientation of the sensing head and/or of the sensing device in spatial orientation and a navigation base station for the spatial recording of the position of the marking means. Further, a scanning data processing unit for producing a corrected skeleton image and/or anatomical rotational points of the body portion based on the primary skeleton image and the depth scan image provided with position data and/or the position data of the bony references scanned with the sensing device.

This system from which individual parts may also be omitted in certain applications and in particular the image data processing unit, respectively, the scanning data processing unit, respectively, the scanning data processing unit can also be replaced by a processing capacity external to the system such as on a PC or PDA, etc., performs accordingly a multi-step generation of an accurate skeleton image (or more generally also depth structure images) of the body portion. In this respect, surface image data from the large area image is combined with depth image data and/or position data of individual skeleton, respectively, structural points are position-accurate fidelity.

In an embodiment of the invention, the sensing head is formed as an ultrasound sensing head and a calibrating unit for calibrating the position data linked to the depth scan image of the sensing head based on its imaging characteristic is associated with the navigation base station. In this embodiment, a well-established and clinically-proven apparatus available in high quality is used for generating depth image data and appropriate means are added to this for assigning the position of the scanned data, respectively, images.

In another embodiment, the generation of depth image data is dispensed with and in its place, defined points of the structure to be examined, particularly prominent bony references are scanned with a sensing device while simultaneously determining the position of the sensing device, only selected position data is superimposed over the image data of the body surface, respectively, the primary skeleton image thereby generated. This enables likewise a precise further process of the structured image, respectively, skeleton images, or corresponding raw data to anatomical rotational points which can satisfy for all intents and purposes certain diagnostic requirements.

Both embodiments mentioned can also be combined and produce in the combination a particularly high degree of image accuracy and diagnostic relevance.

In another embodiment of the invention, marking means (“second marking means”) are integrated in the sensing head and/or sensing device or are firmly connectable with the same. Marking means can hereby be formed through specifically formed surface portions of the sensing head and/or sensing device and the navigation base station designed for recognizing the specifically formed surface portions and for determining their position. It is understood that alternatively or also additionally locators (“tripods”) known per se can be used for this purpose.

In another embodiment, which is particularly useful when employing a sensing device exclusively to refine the originally generated image, separate reference marking pins (“third marking means”) are provided with fixing means for stationary connection with the body. The positions of the sensing device can then be set in relation to the positions of the of the additional marking pins in order to take into account small body movements in the processing calculation for generating the corrected skeleton image. Present marking means are preferably attached at the pelvis of the test persons, e.g. on the back in the region of the sacrum. This can not only serve as reference marker and for compensation of body sway by inputting further points but rather also during a movement measurement which records very small movements of the pelvis. Since the test persons wear clothing, these small movements in the measurement only of the 3D body surface cannot be recorded with sufficient accuracy.

A further advantage of this arrangement with preferably three reference markers is that with the sensing stylus two points at the iliac crest and a point at the iliac in relation to the reference markers can be inputted. Thereby it is possible for a model calculation to determine the location of the hip joint centers left and right and to use these in the animated skeleton.

In a particularly preferred embodiment, these reference markers are connected in a fixed relation to an additional inertial sensor which can also then measure the angle movements of the pelvis when the test person has moved away from the range of the scanning means of the reference marker. The inertial sensor system can transmit its data via radio and is operated simultaneously with the 3D surface recording system. The latter can perform the entire measurement of the pelvic movements even when the test person in the room moves past the 3D camera systems arranged (as described) in pairs on the left and right.

Specifically for the simultaneous scanning of references or joint points corresponding to one another on both arms or both legs, a sensing device formed in the manner of a sensing caliper or a sensing circle can be provided which is provided of course with marking means. The attachment to the body can occur by adhesion, suction pads, or also by means of suitable straps or belts which wrap around the body in the region to be examined and to which additional locators can be suitably affixed.

In further embodiments of the invention, the marking means comprise transmitter or receiver units operating on the basis of ultrasound or on an optical or magneto metric basis, or reflectors and the navigation base station includes for this purpose corresponding receiver and/or transmitter units. Such marking means (locators) and base stations operating in cooperation with them are known per se and are offered, among others, by the Applicant. This applies also for the expedient embodiment of the optical scanning device as structured light scanner. The scanning means, apart from this, can be executed as a laser scanner or a camera means, in particular a 3D-camera, is used.

Whereas in certain embodiments of the invention the components for determining the position are provided as a separate sub system in addition to the large area imaging apparatus, in a special embodiment the scanning means is arranged and designed such that it functions at the same time as a navigation base station for determining the position of the marking means. This simplifies the overall technical configuration of the device and the handling. In accordance with the above-mentioned system design additional references can be provided thereby in particular first marking means on a handheld sensing stylus and second marking means serving as reference marking means for the temporary fixed connection with the body. When using an optical scanning means corresponding optical markers are used while a large area operating ultrasound scanning means requires of course the use of ultrasound transmitting elements or reflectors.

In a further arrangement of the proposed apparatus, especially for the analysis of the gait of a human being, it is provided that the large area imaging apparatus comprise respectively at least one 3D camera mounted on both sides of the vertebrate for imaging its arms and/or legs or portions thereof, wherein the cameras on the right and left side include means for generating a defined positional relationship between them. More specifically, this embodiment is designed such that on the right and left sides of a predetermined direction of movement of the vertebrate, a plurality of 3D cameras is positioned, respectively, in predetermined intervals. A further embodiment provides that in addition to the 3D cameras on the right and left sides, a 3D camera is mounted at the rear side of the vertebrate or a portion thereof which preferably comprises also means for generating a defined positional relationship to the cameras on the right and left sides.

When using a sensing head for scanning a depth image of the examined body portion, specifically an ultrasound sensing head, a relationship as precise as possible has to be established between the determinable position of the sensing head and the spatial position of the depth image generated by it. The calibration unit mentioned further above serves this purpose and the data required are scanned in an embodiment of the invention by means of a special calibrating device as a device component.

### BRIEF DESCRIPTION OF THE DRAWINGS

Advantages and expedient embodiments of the invention are also inferable from the following description of the exemplary embodiments and aspects by means of the figures. In the figures:

FIG. 1 shows a complete overview of an embodiment of the apparatus according to the invention in the form of a block diagram,

FIGS. 2A and 2B show illustrations to elucidate the function of the apparatus according to FIG. 1,

FIG. 3 shows a perspective view of a structured light projector with an integrated navigation base station as it can be used in a similar apparatus as shown in FIG. 1,

FIG. 4 shows a perspective view of an ultrasound sensing head with marking means for use in the apparatus according to FIG. 1 and in a similar apparatus,

FIG. 5 shows a schematic perspective illustration of a further embodiment of the apparatus according to the invention, and

FIGS. 6A and 6B show a sketch-like representation of further embodiments of the invention.

### DETAILED DESCRIPTION OF THE PREFERRED EMBODIMENTS

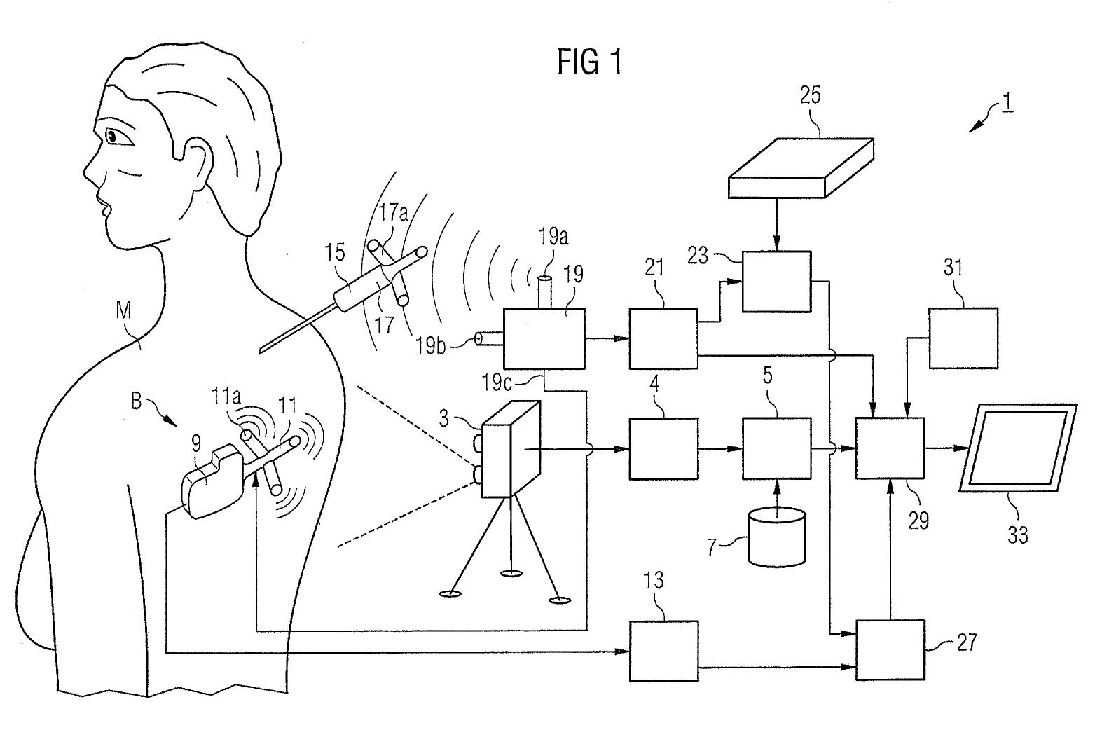

FIG. 1 shows schematically the structure of an imaging apparatus 1 for the large area imaging of a body section B of a human being M according to an embodiment of the invention. A stationary structured light scanner 3 arranged opposite the human being M generates by scanning an overall image of the surface of the back B, respectively, a set of corresponding image data and transfers this to a pre-processing unit 4 where a surface image of the back B is synthesized. This image arrives at an image data processing unit 5 to generate a primary skeleton image of the bone structure of the back B by accessing standard skeleton images, respectively, corresponding anatomical data stored in a database 7.

Furthermore, the apparatus represented includes an ultrasound scanning head 9 with integrated locator 11 with three ultrasound transmitting units 11a whose data output is connected with an ultrasound image generating unit 13. In addition, a sensing device 15 with integrated locator 17 which (in contrast to locator 11 of the ultrasound sensing head 9) as marking elements bear ultrasound reflectors 17a. Generally, the use of sensing device 15 with integrated locator requires the additional attachment of reference marking means on the body of the patient in order to obtain more refined position determination of specific anatomically prominent (as explained above). For reasons of clarity, such reference marking means are not shown in the figure. With respect to their execution and attachment, reference can be made to DE 196 49 399 A1, for example.

A navigation base station 19 cooperates with the locators 11 and 17 which comprises, on the one hand, via an ultrasound transmitter 19A for emitting ultrasound signals to locator 17 and on the other hand, via an ultrasound receiver 19b for receiving ultrasound signals from the sender elements 11a of the locator 11, respectively, reflected ultrasound signals from the reflector elements 17a of the locator 17. Furthermore, the navigation base station 19 has a cable connection 19c to locator 11 in order to supply and to control in appropriate synchronization its sender element 11a. The spatial position of the navigation base station 19 in respect of the structured light scanner 3 has to be defined in order to be able to correctly correlate the position data with the image data of the surface of the back. The appropriately synchronized generation and ordered processing of the sender signals or control signals on the one hand and the received signals, on the other hand, is performed in the navigation base station fundamentally in the known manner so that a corresponding description is not required here.

The pre-processed received signals in the navigation base station 19 reach a position data determination unit 21 which determines the spatial position of the ultrasound sensing head 9, respectively, sensing device 15. To the extent that position data of the ultrasound sensing head 9 are concerned, the data are presented to a calibrating unit 23 which receives data in addition from a calibrating device 23 and delivers calibrated position data which can be directly associated with the depth scan image generated by means of the ultrasound sensing head 9 of back B. This association occurs in an ultrasound data linking unit 27. The position data related to the sensing device 15, in contrast, do not undergo any additional processing.

The calibrating device 25 serves for delivering data which characterize the imaging characteristic of the sensing head 9 in which a conversion is performed of the scanned position data of the sensing head into position data to be associated with the ultrasound image. An embodiment of the calibrating device can appear such that on a holding plate fixed marking points (ultrasound senders) as well as a water bath are attached, into which the sensing head is submerged and in which the sensing head is put into operation for the purpose of calibration. The generated ultrasound image is recorded and the sensing head via setting screws is adjusted until an inserted metal pin in the water bath can be clearly recognized in fixed spatial relationship to the ultrasound senders in the ultrasound image (sectional view). Through the determination of the position data of the ultrasound sender serving as reference here and the known spatial relationship between the metal pin and the ultrasound senders of the calibrating device, a sufficiently exact relationship can be established between the position data of the sensing head and the ultrasound image, respectively, the depth image data.

The image data of the primary skeleton image from the image data processing unit 5 are consolidated with the position data of the sensing device 15 and the calibrating position data of the ultrasound images (depth scan images) in a sensing data processing unit 29. Into the latter enter also inputs from an input unit 31, for example for designating an ongoing scanned bony reference with the sensing device 15 in the body portion (back) B. To obtain the desired refined (corrected) skeleton image, the bony edge contours are recognized from the ultrasound images through well-known algorithms and by means of known software. The ultrasound images can also be correspondingly manually processed by the physician. A complete scanning of the back with the sensing head, moreover, is not required.

In the sensing data processing unit 29, a corrected skeleton image of the back is computed based on all of these data and this can be displayed on a display unit 33, It is understood that also the images obtained as a result of different pre-processing steps can be displayed, corresponding signal connections are, however, not indicated in the drawing in the interest of clarity.

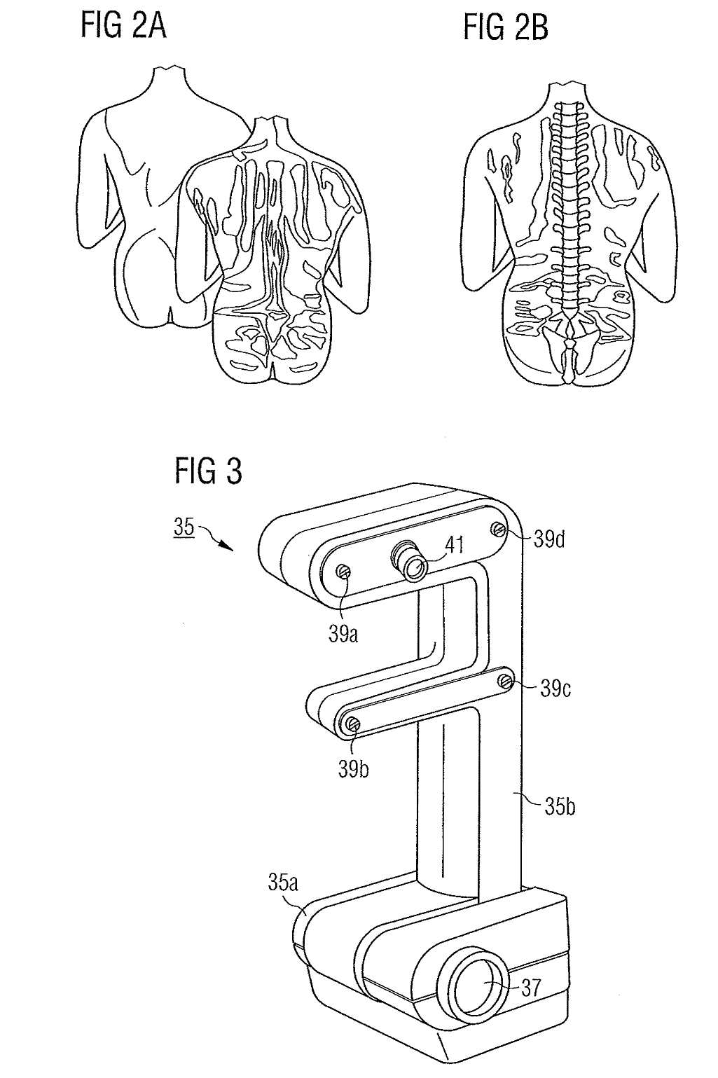

FIGS. 2A and 2B show for the purpose of illustration the mode of operation of apparatus 1, on the one hand, an image of the body surface together with a “curvature map” of the back which serves to visualize anatomical orientation points (landmarks), and on the other hand, a skeleton image, respectively, a depth structure image as can typically occur at the end of the processing process of the apparatus.

FIG. 3 shows a combination apparatus 35 which combines the functional components of a structured light scanner as well as a navigation base station and in a similar apparatus as in the one shown in FIG. 1, could generally replace the components structured light scanner 3 and the navigation base station 19. On a base body 35a which receives a structured light projector 37, a stand-like extension 35b is mounted in which two superimposed platforms bear on the one hand four ultrasound receivers 39a to 39d (which can not be recognized separately) of a navigation base station as well as a camera 41 in addition. Since both the mode of operation of a structured light scanner as well as that of a navigation base station of the type used here as well as those of a camera is known, a further detailed description is not necessary.

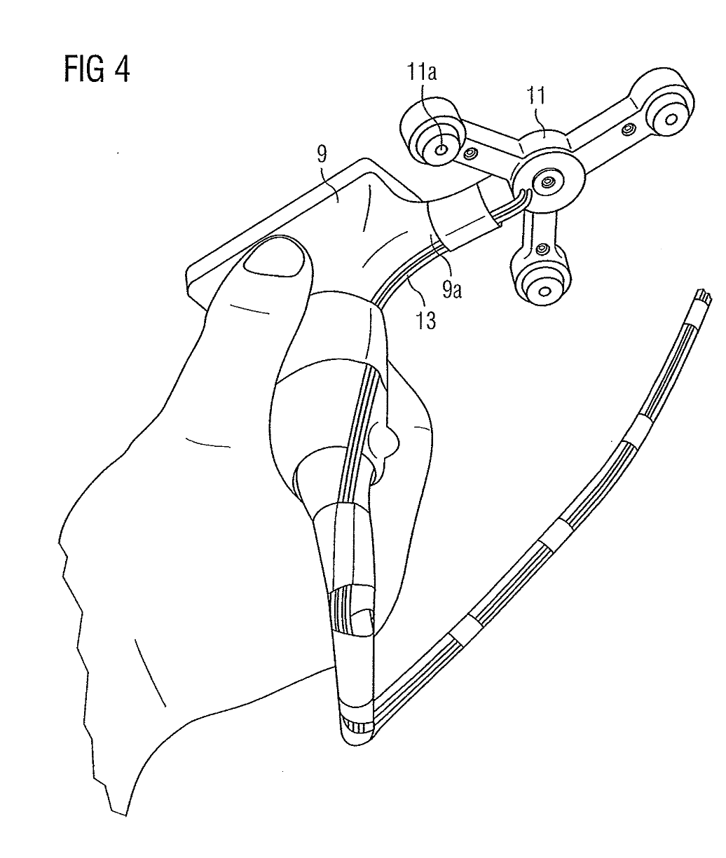

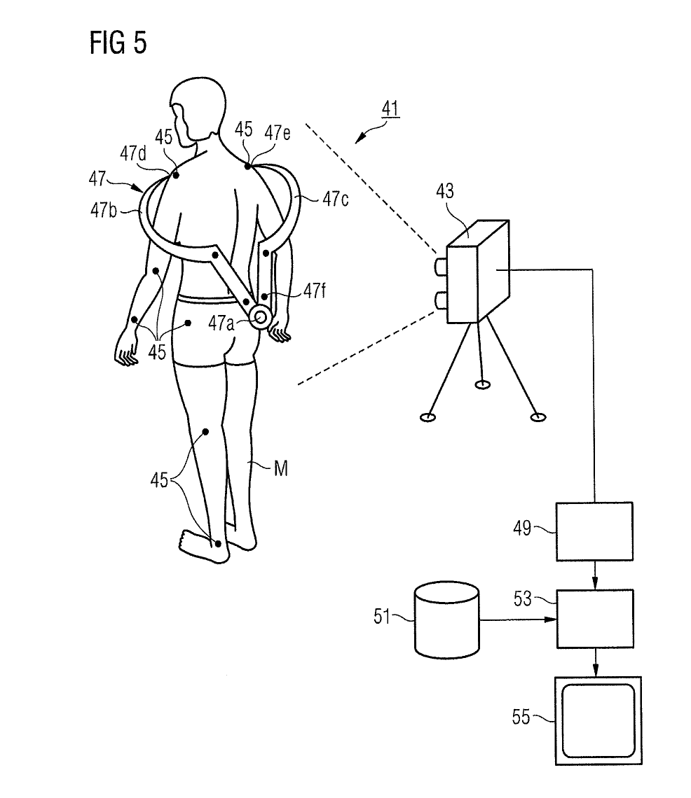

FIG. 4 shows in a perspective illustration an embodiment of the ultrasound sensing head 9 with inserted locator 11 more exactly. It can be seen that the locator 11 with the navigation means 11a (which generally can be receiver or sender or also reflectors and can cooperate with appropriate corresponding sender, respectively, receiver elements in a navigation base station) is mounted on an extension 9a of the sensing housing that the handling by the physician does not hinder the ultrasound transmission. Connection cables 13 of locator 11 are guided as far as possible alongside the housing of the sensing device 9.

FIG. 5 shows in a sketch-like manner a further imaging apparatus 41 according to the invention in use on a human test person M, wherein the imaging apparatus includes a 3D camera 43 as large area imaging apparatus and is especially designed for determining anatomical rotational points 45 of a human body and for its anatomically largely correct representation in motion. The anatomical rotational points concern, as can be derived from the drawing, in particular the hip joint as well as knee, ankle, foot as well as shoulder and arm joints of the test person M. These rotational points 45 cannot be sufficiently recorded from a simple 3D surface image with a standing test person—especially with test persons with restricted mobility, or the determination is very complicated. Additional difficulties arise when clothing causes an additional lack of definition of the contour of the body.

Possible and useful here is a mechanical scanning connected with a “navigation”. For this purpose, a previously mentioned sensing stylus can be used; in the figure, however, a sensing caliper 47 as an especially constructed sensing device is illustrated with which in each case the respective corresponding right- and left-sided rotational points 45 can be scanned at the same time. The sensing caliper 47 includes two pivotal arms 47b, 47c of the same lengths around a rotational axis 47a, located on their free ends in each case is a sensing tip 47d, 47e. Navigation marking points 47f installed on the arms 47b, 47c, navigation marking points 47f enable the determination of the respective spatial position of the sensing caliper, optionally by a separate (in the drawing not illustrated) conventional position determining system or in simple manner directly by means of the 3D camera 43. From the spatial coordinates of the marking points 47f determined hereby, the spatial coordinates of the sensing tips 47d, 47e can be determined by simple offset calculation and thus the position of the respectively scanned rotational points of the body.

On the basis of these spatial coordinates, a virtual joint-related skeleton model of the test person M can be generated in a processing unit 49 and in further processing steps the measured 3D surface data can be optimally brought to match continuously with skeleton data determined in advance from a skeleton data base 51, respectively, the measured coordinates of the rotational points 45. The skeleton database 51 can contain data sets in particular which are obtained in advance in the manner described further above with reference to FIG. 1. Therefore, a 3D model of the test person (e.g. as skeleton model) can finally be computed in a further processing step 53 and shown in motion on a display monitor 55, i.e., the movements of the test person can be shown in visual images solely due to the continuous surface data obtained with the 3D camera 43.

In a further embodiment of the method, moreover, an imaging of the measurement of the body surface can also be performed during the input/recording of anatomical rotational points with the sensing caliper 47 (or also with a simple sensing stylus which is relatively more time-consuming). In this connection, changes in the position of the rotational points during the imaging, respectively, recording procedure can be taken into account and, if applicable, corrected. Such changes in position can occur, for example, in that the test person sways slightly in the time period required or involuntarily executes movements.

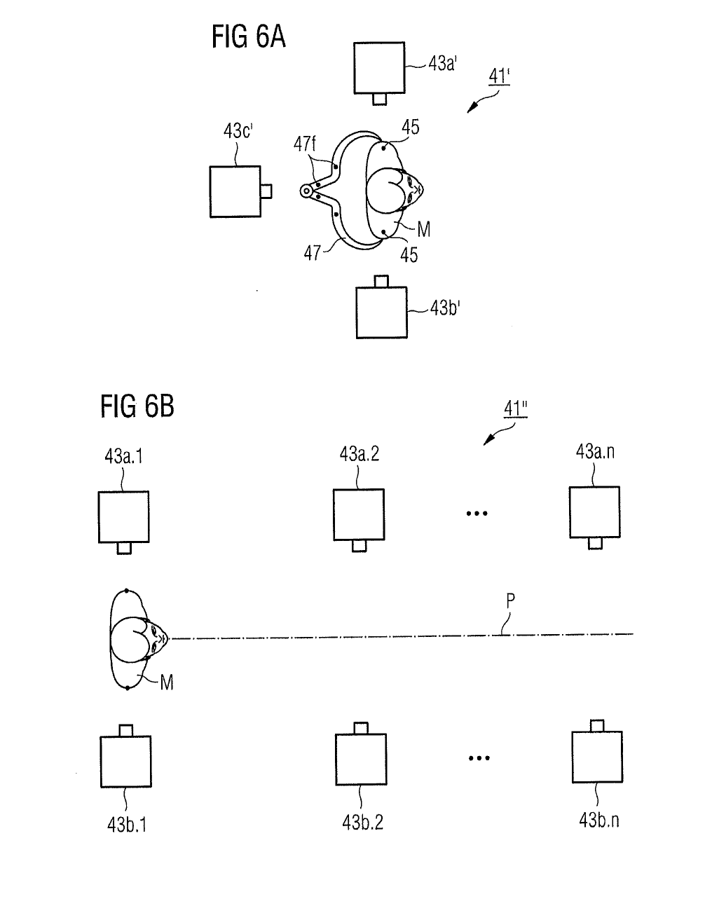

In the FIGS. 6A and 6B, sketch-like modifications of the apparatus shown in FIG. 5 is depicted. FIG. 6A shows hereby an imaging apparatus 41′ in which a group of three 3D cameras 43 a′, 43b′ and 43c′ is provided as a large area imaging apparatus. Cameras 43a′, 43b′ thereof are positioned on the right, respectively, left side of the test person M while the third camera 43c′ is at his back. As in the remarks concerning FIG. 5, anatomical rotational points 45 are scanned by means of the sensing caliper 47 and the markers 47f thereof are recorded either by the left or right or by both lateral cameras.

The camera 43c′ at the rear side (the provision of which is optional) can also record the markers 47f alone during an initial determination of spatial coordinates of the anatomical rotational points, and the lateral cameras then take over the recording of movements of the test person. These lateral cameras could then be only 3D imaging means while the camera on the rear side could be designed and function specifically as a camera of a position determination system. To the extent a further (not shown) scanning means of a position determining system for recording marker signals is provided, of course also all three cameras can be simple surface imaging apparatus.

In the arrangement just described, leg and arm movements perhaps can be captured more readily. Important is that, in particular, the laterally arranged cameras 43a′, 43b′ are arranged in a defined relative position to each other so that a defined measuring space is spanned by both cameras. In the simplest case, only the distance for this between the two cameras is determined; a preferred embodiment provides, however, a calibrated definition of the specified measuring space through the evaluation of the signals of markers which lie in the field of vision of both cameras.

The other modified imagining apparatus 41″ which is sketched in FIG. 6B, includes, respectively, an entire group of cameras 34a.1 to 34a.n on the left side and cameras 34b.1 to 34b.n on the right side, which in turn are positioned in a predetermined positional relationship to one another along a movement path P of the test person M. This further arrangement is suitable for analyzing the gait of the test person over a longer distance. It is understood that with this further design calibration is also required to determine a defined measuring space. Such determinations/calibrations are known per se to the person skilled in the art for position determination systems and consequently are not further described here.

The execution of the invention is not limited to above-described examples and emphasized aspects, but is also possible in a plurality of modifications lying within the skills of the competent skilled person. Specifically, it is pointed out once again that instead of a conventional structured light scanner, a laser scanner or a camera means with appropriate performance parameters can also be provided and that the optical scanning means with suitable means for image processing as a prerequisite, can be designed at the same time as a navigation base station and in this case cooperates with optically functioning marking means (if applicable, also clearly defined housing portions of the sensing head and/or sensing device) and can process these to obtain position data.

1. An imaging apparatus for large area imaging of a body portion of a vertebrate, comprising navigation means for determining a position of imaged body points, wherein the imaging apparatus comprises:
an optical scanning means for generating image data of a body surface, an image data processing unit for generating a primary skeleton image of bone and/or joint structure of the body portion from the image data of the body surface and stored standard skeleton images,
at least one of a sensing head for stepwise scanning of a depth scan image of the body portion or a sensing device for sensing bony references in the body portion,
marking means for marking a position and orientation of the at least one of the sensing head or the sensing device for spatial orientation and a navigation base station for spatial recording of a position of the marking means, and
a scanning data-processing unit for producing a corrected skeleton image and/or anatomical rotational points of the body portion based on the primary skeleton image and on the depth scan image provided with at least one of the position data or the position data of the bony references scanned with the sensing device.

2. The imaging apparatus according to claim 1, wherein the sensing head is formed as an ultrasound sensing head and a calibrating unit for calibrating the depth-scan image associated position data of the sensing head based on imaging characteristics of the sensing head is associated with the navigation base station.

3. The imaging apparatus according to claim 1, wherein first marking means are integrated in the at least one of the sensing head or sensing device or are firmly connected thereto.

4. The imaging apparatus according to claim 3, wherein first marking means are formed through specifically formed surface sections of the at least one of the sensing head or sensing device, and the navigation base station is adapted to recognize specifically formed surface sections and to record their position.

5. The imaging device according to claim 1, wherein separate reference marking pens are provided with fixing means for stationary connection with the body.

6. The imaging apparatus according to claim 1, wherein the marking means comprise transmitting or receiving units or reflectors operating on an ultrasound or on an optical or magneto metrical basis and the navigation base station includes for this purpose at least one of a corresponding receiver or transmitting unit.

7. The imaging apparatus according to claim 1, wherein the optical scanning means comprises a structured light scanner or laser scanner or a camera apparatus.

8. The imaging apparatus according to claim 1, wherein the optical scanning means or a component thereof is arranged away from the body and is adapted to function simultaneously with the navigation base station for determining the optical position of the marking means.

9. The imaging apparatus according to claim 7, wherein the optical scanning means comprises respectively at least one 3D camera located on both sides of the vertebrate for imaging arms and/or legs or portions thereof, wherein cameras of the at least one 3D camera located on right and left sides comprise means for establishing a defined positional relationship between them.

10. The imaging apparatus according to claim 9, wherein on the right and left side of a predetermined direction of movement of the vertebrate, respectively, a plurality of 3D cameras are placed in predetermined intervals.

11. The imaging apparatus according to claim 9, wherein in addition to the 3D cameras on the right and left side, on a rear side a 3D camera is mounted for imaging a rear side of the vertebrate or a portion thereof.

12. The imaging apparatus according to claim 8, wherein second marking means on a handheld sensor pen or on a handheld sensor caliper and third optical marking means serving as reference marking means are provided for temporary fixed connection with the body.

13. The imaging apparatus according to claim 2, further comprising a calibrating device for generating imaging characteristics used in the calibrating unit of the ultrasound sensing head.

14. An imaging apparatus for large area imaging of a body portion of a vertebrate, comprising navigation means for determining a position of imaged body points, wherein the imaging apparatus for imaging moving images comprises:
an optical scanning means for generating image data of a body surface,
a first image data processing unit for generating primary data of bone and joint structure of the body portion from the image data of the body surface and stored standard skeleton images,
at least one of a sensing head for step-wise scanning of a depth scan image of the body portion or a sensing device for sensing bony references in the body portion,
marking means for marking a position and orientation of the at least one of the sensing head or the sensing device in spatial orientation and a navigation base station for the spatial recording of the position of the marking means, and
a second image data processing unit for generating an animated skeleton image, including dynamic determination of anatomical rotational points of the body portion based on primary data and the depth scan image provided with at least one of position data or position data of bony references scanned with the sensing device.

15. The imaging apparatus according to claim 14, wherein the sensing head is designed as an ultrasound sensing head and a calibrating unit is associated with the navigation base station for calibrating the position data associated with the depth scan image of the sensing head on a basis of imaging characteristics of the sensing head.

16. The imaging apparatus according to claim 14, wherein first marking means are integrated in the at least one of the sensing head or sensing device or are firmly connectable therewith.

17. The imaging apparatus according to claim 16, wherein first marking means are formed by specifically formed surface portions of the at least one of the sensing head or the sensing device and the navigation base station is adapted to record the specifically formed surface portions and to determine their position.

18. The imaging apparatus according to claim 14, wherein separate reference marking pins are provided with fixation means for stationary connection to the body for compensatory signal processing to compensate for involuntary body movement during a scanning step.

19. The imaging apparatus according to claim 14, wherein the marking means comprise transmitting or receiving units operating on an ultrasound or on an optical or magneto metrical basis or reflectors, and the navigation base station includes for this purpose at least one of a corresponding receiving or transmitting unit.

20. The imaging apparatus according to claim 14, wherein the optical scanning means comprises a structured light scanner or laser scanner or a camera apparatus.

21. The imaging apparatus according to claim 14, wherein the optical scanning means or a component thereof is arranged away from the body and is adapted to function simultaneously with the navigation base station to optically determine a position of the marking means.

22. The imaging apparatus according to claim 20, wherein the optical scanning means comprises respectively at least one 3D camera mounted on both sides of the vertebrate for imaging its arms and/or legs or portions thereof, wherein cameras of the at least one 3D camera on right and left sides comprise means for establishing a defined positional relationship between them.

23. The imaging apparatus according to claim 22, wherein a plurality of 3D cameras is mounted, respectively, in predetermined intervals on the right and left side of a predetermined direction of movement of the vertebrate.

24. The imaging apparatus according to claim 22, wherein in addition to the 3D cameras on the right and left sides, on a rear side a 3D camera is mounted for imaging the rear side of the vertebrate or a portion thereof.

25. The imaging apparatus according to claim 21, wherein second optical marking means on a hand-held scanning pen or on a hand-held scanning caliper and third optical means, as reference marking means for compensatory signal processing to compensate involuntary body movements during a scanning step are provided for temporary fixed connection with the body.

26. The imaging apparatus according to claim 16, further comprising a calibrating device for generating imagining characteristics of the ultrasound sensing head to be used in the calibrating unit.
